(19) United States
(12) Patent Application Publication
(10) Pub. No.: US 2023/0141868 A1
(43) Pub. Date: May 11, 2023

(54) MICROORGANISM FOR IMPROVING LIVER FUNCTION OR INHIBITING FAT ACCUMULATION, AND USES OF SAME

(71) Applicant: MEDYTOX INC., Cheongju-si, Chungcheongbuk-do (KR)

(72) Inventors: Woo Jin CHOI, Suwon-si, Gyeonggi-do (KR); Tai Hoon KIM, Incheon (KR)

(21) Appl. No.: 17/913,505

(22) PCT Filed: Nov. 17, 2020
(86) PCT No.: PCT/KR2020/016195

§ 371 Date: Sep. 22, 2022

(30) Foreign Application Priority Data
Mar. 24, 2020 (KR) 10-2020-0035900

(51) Int. Cl. A61K 35/747; C12N 1/20; A61P 1/16; A23L 33/135

(52) U.S. Cl. CPC A61K 35/747; C12N 1/205; A61P 1/16; A23L 33/135; C12R 2001/225

(57) ABSTRACT

Provided are a microorganism selected from the group consisting of Lactobacillus salivarius LMT15-14 (Accession No. KCTC14142BP) and Lactobacillus plantarum LMT19-1 (Accession No. KCTC14141BP) or a combination thereof or a culture or extract thereof, and a use thereof.

### TECHNICAL FIELD

The present disclosure relates to a microorganism selected from the group consisting of Lactobacillus salivarius LMT15-14 (Accession No. KCTC14142BP) and Lactobacillus plantarum LMT19-1 (Accession No. KCTC14141BP) or a combination thereof or a culture or extract thereof, and a use thereof.

### BACKGROUND ART

Lactobacillus is a genus of Gram-positive, facultative anaerobic or microaerophilic, rod-shaped, non-spore-forming bacteria. Lactobacillus is a major part of the lactic acid bacteria group. In humans, Lactobacillus is a major component of the microbiota in many parts of a body, such as a digestive system, urinary system, and reproductive system.

The bacteria in the genus Lactobacillus are found in foods such as yogurt. In addition, some Lactobacillus species have physiological activity, such as anti-inflammatory activity. For example, some Lactobacillus has been reported to be effective in irritable bowel syndrome (IBS).

On the other hand, obesity indicates an excessive accumulation of fat in a body. Obesity is known as a cause of diseases such as fatty liver, hyperlipidemia, hyperglycemia, atherosclerosis, and diabetes. Obesity appears as a result of an increase in the number of adipocytes and an increase in the lipid content of adipocytes as a result of adipogenesis. Adipocytes play a major role in synthesizing and storing excess calories as triglycerides, and as a result of adipogenesis, the size and number of adipocytes increase, and accumulation of lipids in cells is accelerated.

Fatty liver is caused by accumulation of excess fat in the liver, which is generally diagnosed as fatty liver when fat is accumulated more than 5% of the weight of the liver. Such fatty liver may be divided into alcoholic fatty liver due to excessive drinking and non-alcoholic fatty liver that occurs independently of alcohol. Non-alcoholic fatty liver disease is more than just one disease, and includes a variety of diseases, from mild fatty liver to chronic hepatitis to cirrhosis. Non-alcoholic fatty liver is associated with metabolic syndromes such as obesity, adult diabetes, and hyperlipidemia. When excessive calories are continuously consumed, fat accumulates in adipocytes in the body and in the liver, and the increased fat may cause secretion of various substances that are harmful to the liver, e.g., cytokines, and induce steatohepatitis and cirrhosis.

Bacteria in the genus Lactobacillus are a major member of the normal microbiota that inhabit the human gut, and have long been known to be important in maintaining a healthy digestive system and vaginal environment. According to the U.S. Public Health Service guidelines, all of the Lactobacillus strains currently deposited with the U.S. American Type Culture Collection (ATCC) are classified as Bio-Safety Level 1, which means that all of the Lactobacillus strains are recognized that nothing is known about the potential risk of causing disease to humans or animals.

However, lactic acid bacteria are known to have excellent immune response modulating effects and anti-cancer and antioxidant effects through existing studies, but not much is known about the effect of Lactobacillus strains on reducing the fat content in the body or the effect of treating fat-related diseases.

### DESCRIPTION OF EMBODIMENTS

### Technical Problem

A goal is to provide a microorganism selected from the group consisting of Lactobacillus salivarius LMT15-14 (Accession No. KCTC14142BP) and Lactobacillus plantarum LMT19-1 (Accession No. KCTC14141BP), each having activity of inhibiting triglycerides, promoting fat oxidation, and inhibiting liposynthesis, or a combination thereof.

Another goal is to provide a pharmaceutical composition, for improving liver function or preventing or treating obesity-related disease, containing the microorganism or a culture or extract thereof, or mixtures thereof, as an active ingredient.

Another goal is to provide a food composition, for improving liver function or preventing or improving obesity-related disease, containing the microorganism or a culture or extract thereof, or mixtures thereof, as an active ingredient.

### Solution to Problem

An aspect provides a microorganism selected from the group consisting of Lactobacillus salivarius LMT15-14 (Accession No. KCTC14142BP) and Lactobacillus plantarum LMT19-1 (Accession No. KCTC14141BP), each having activity of inhibiting triglycerides, promoting fat oxidation, and inhibiting liposynthesis, or a combination thereof.

Another aspect provides a microorganism selected from the group consisting of Lactobacillus salivarius LMT15-14 (Accession No. KCTC14142BP) and Lactobacillus plantarum LMT19-1 (Accession No. KCTC14141BP) or a combination thereof, or a culture or extract thereof. The extract may be a protein extract of a microorganism or a combination thereof. The extract may be a lysate obtained by lysing the microorganism or a combination thereof, or a remaining supernatant from which a precipitate is removed after centrifugation of the lysate.

In the microorganism or a combination thereof, or a culture or extract thereof, the combination may be a mixture of Lactobacillus salivarius LMT15-14 (Accession No. KCTC14142BP) and Lactobacillus plantarum LMT19-1 (Accession No. KCTC14141BP) at any ratio by weight. For example, the mixing ratio may be from 1:0.3 to 3.0.

The microorganism or a combination thereof, or a culture or extract thereof may be at least one selected from the group consisting of one having acid resistance, one having bile resistance, one having activity of promoting oxidation, one having activity of inhibiting liposynthesis, or one having the aforementioned two activities, one inhibiting fat accumulation or reducing accumulated fat, one inhibiting gene expression of at least one selected from the group consisting of SREBP-1c and FAS, one promoting gene expression of at least one selected from the group consisting of PPAR-1α and CPT1, one increasing a phosphorylation level of AMPK, one increasing a level of adiponectin in blood when administered to an individual, one decreasing at least one selected from the group consisting of body weight and an amount of fat in a body when administered to an individual, and one improving liver function. The fat may be triglycerides.

The acid resistance may be viability of at least 80%, at least 85%, at least 90%, 80% to 90%, 80% to 95%, 85% to 90%, or 90% to 95%, when incubated for 2 hours at pH 2.5 and 37° C. in MRS medium.

The bile resistance may be viability of at least 75%, at least 80%, at least 90%, at least 95%, 75% to 90%, 75% to 95%, 80% to 90%, 80% to 95%, 85% to 90%, or 90% to 95%, when incubated for 2 hours at 37° C. in MRS medium containing 0.3% bile acid.

The microorganism or a combination thereof, or a culture or extract thereof may promote expression of at least one gene selected from the group consisting of PPAR-1α and CPT1. The promotion of the gene expression may increase gene expression of at least one gene selected from the group consisting of PPAR-1α and CPT1, as compared with absence thereof, by at least 10%, at least 15%, at least 20%, at least 25%, at least 30%, at least 35%, at least 45%, at least 50%, at least 55%, at least 65%, at least 70%, at least 75%, at least 80%, at least 85%, at least 90%, at least 100%, 10% to 100%, 20% to 100%, 30% to 100%, 40% to 100%, 50% to 100%, 60% to 100%, 70% to 100%, 80% to 100%, or 90% to 100%.

The microorganism or a combination thereof, or a culture or extract thereof may inhibit expression of at least one gene selected from the group consisting of SREBP-1c and FAS. The inhibition of the gene expression may reduce gene expression of at least one gene selected from the group consisting of SREBP-1c and FAS, as compared with absence thereof, by at least 10%, at least 15%, at least 20%, at least 25%, at least 30%, at least 35%, at least 45%, at least 50%, at least 55%, at least 65%, at least 70%, at least 75%, at least 80%, at least 85%, at least 90%, at least 100%, 10% to 100%, 20% to 100%, 30% to 100%, 40% to 100%, 50% to 100%, 60% to 100%, 70% to 100%, 80% to 100%, or 90% to 100%.

The microorganism or a combination thereof, or a culture or extract thereof may reduce an amount of fat or inhibit accumulation of fat. The inhibition of the accumulation of fat may reduce an amount or accumulation of fat, as compared with absence thereof, by at least 10%, at least 15%, at least 20%, at least 25%, at least 30%, at least 35%, at least 45%, at least 50%, at least 55%, at least 65%, at least 70%, at least 75%, at least 80%, at least 85%, at least 90%, at least 100%, 10% to 100%, 20% to 100%, 30% to 100%, 40% to 100%, 50% to 100%, 60% to 100%, 70% to 100%, 80% to 100%, or 90% to 100%.

The microorganism or a combination thereof, or a culture or extract thereof may reduce at least one selected from the group consisting of a body weight and an amount of adipose tissue in a body when administered to an individual. The reduction at least one of the body weight and an amount of adipose tissue may be reducing, as compared with absence thereof, by at least 10%, at least 15%, at least 20%, at least 25%, at least 30%, at least 35%, at least 45%, at least 50%, at least 55%, at least 65%, at least 70%, at least 75%, at least 80%, at least 85%, at least 90%, at least 100%, 10% to 100%, 20% to 100%, 30% to 100%, 40% to 100%, 50% to 100%, 60% to 100%, 70% to 100%, 80% to 100%, or 90% to 100%.

The microorganism or a combination thereof, or a culture or extract thereof may reduce a level of triglycerides. The reduction of the level of triglycerides may be reducing, as compared with absence thereof, by at least 10%, at least 15%, at least 20%, at least 25%, at least 30%, at least 35%, at least 45%, at least 50%, at least 55%, at least 65%, at least 70%, at least 75%, at least 80%, at least 85%, at least 90%, at least 100%, 10% to 100%, 20% to 100%, 30% to 100%, 40% to 100%, 50% to 100%, 60% to 100%, 70% to 100%, 80% to 100%, or 90% to 100%, based on weight.

Another aspect provides a composition including the microorganism selected from the group consisting of Lactobacillus salivarius LMT15-14 (Accession No. KCTC14142BP) and Lactobacillus plantarum LMT19-1 (Accession No. KCTC14141BP) ora combination thereof, or a culture or extract thereof, as an active ingredient.

The composition may be at least one selected from the group consisting of one having acid resistance, one having bile resistance, one having activity of promoting oxidation, one having activity of inhibiting liposynthesis, or one having the aforementioned two activities, one inhibiting fat accumulation or reducing accumulated fat, one inhibiting gene expression of at least one selected from the group consisting of SREBP-1c and FAS, one promoting gene expression of at least one selected from the group consisting of PPAR-1α and CPT1, one increasing a phosphorylation level of AMPK, one increasing a level of adiponectin in blood when administered to an individual, one decreasing at least one selected from the group consisting of body weight and an amount of fat in a body when administered to an individual, and one improving liver function. The fat may be triglycerides.

Therefore, as the composition has acid resistance and bile resistance, the composition may be used in an acidic intestine. In addition, the composition may be used in at least one selected from the group consisting of promoting oxidation, inhibiting liposynthesis, reducing an amount or accumulation of fat or reducing accumulated fat, inhibiting gene expression of at least one selected from the group consisting of SREBP-1c and FAS, promoting gene expression at least one selected from the group consisting of PPAR-1α and CPT1, increasing a phosphorylation level of AMPK, increasing a level of adiponectin in blood when administered to an individual, improving liver function, and decreasing at least one selected from the group consisting of body weight and an amount of fat in a body when administered to an individual. The fat may be triglycerides.

Reducing an amount of fat in a body may include reducing an amount of fat for therapeutic purposes. For example, reducing the amount of fat in a body may be intended for use in prevention or treatment of obesity-related diseases. The obesity-related disease may be at least one selected from the group consisting of fatty liver, type 2 diabetes, hyperlipidemia, cardiovascular disease, atherosclerosis, lipid-related metabolic syndrome, and obesity. The fatty liver may be non-alcoholic fatty liver. The individual may be an animal, including human, who has developed or is likely to develop an obesity-related disease.

The composition may be a food composition or a pharmaceutical composition, that is, a medicine. The composition may include a sitologically or pharmaceutically acceptable carrier.

The composition may also include the microorganism or a combination thereof, or a culture or extract thereof in the composition as a “sitologically effective amount” or a “therapeutically effective amount”. With regard to the composition, a “therapeutically effective amount” refers to a sufficient amount which is therapeutically effective upon administered to an individual in a need of treatment. The term “treatment” means to treat a disease or medical condition, such as an obesity disease, in an individual, for example, a mammal, including human, which includes: (a) prevention of occurrence of a disease or medical condition, i.e., prophylactic treatment of a patient; (b) alleviation of a disease or medical condition, i.e., removal or recovery of a disease or medical condition from a patient; (c) suppression of a disease or medical condition, that is, slowing or stopping progression of a disease or medical symptom in an individual; or (d) alleviation of a disease or a medical condition in an individual. The “effective amount” may be properly selected by one of ordinary skill in the art. The “effective amount” may be present at 0.01 mg to 10,000 mg, 0.1 mg to 1,000 mg, 1 mg to 100 mg, 0.01 mg to 1,000 mg, 0.01 mg to 100 mg, 0.01 mg to 10 mg, or 0.01 mg to 1 mg.

The composition may be administered orally. Accordingly, the composition may be formulated in a variety of forms, such as a tablet, a capsule, an aqueous solution, or a suspension. An excipient, such as lactose or corn starch and a lubricating agent, such as magnesium stearate may be commonly added to a parenteral tablet. In the case of parenteral capsule, lactose and/or dried corn starch may be used as a diluent. In case there is a need of an aqueous suspension for the parenteral administration, active ingredients may be combined with an emulsifier and/or a suspension. If needed, a certain sweetening agent and/or a flavoring agent may be added thereto.

Another aspect provides a method of reducing fat content in liver or fat cells, the method including contacting the liver or fat cells with the microorganism selected from the group consisting of Lactobacillus salivarius LMT15-14 (Accession No. KCTC14142BP) and Lactobacillus plantarum LMT19-1 (Accession No. KCTC14141BP) or a combination thereof, or a culture or extract thereof.

In the method, the contacting may be culturing the microorganism or a combination thereof, or a culture or extract thereof in a medium including liver cells or fat cells. The method may be an in vitro or in vivo method.

Another aspect provides a method of reducing fat content or improving liver function in an individual, the method including administering to the individual the microorganism selected from the group consisting of Lactobacillus salivarius LMT15-14 (Accession No. KCTC14142BP) and Lactobacillus plantarum LMT19-1 (Accession No. KCTC14141BP) or a combination thereof, or a culture or extract thereof.

In the method, one of ordinary skill in the art may properly select a route of administration at the time of administration according to the patient's condition. The administration may be an oral or local administration.

In the method, the dosage varies depending on various factors such as the patient's condition, route of administration, the judgment of the attending physician, and the like as described above. Effective dosages may be estimated from dose-response curves obtained in in vitro or animal model tests. The ratio and concentration of the compounds of the present disclosure present in the composition to be administered may be determined according to chemical properties, route of administration, therapeutic dosage, and the like. The dosage may be administered to an individual in an effective amount of from about 1 μg/kg to about 1 g/kg per day, or from about 0.1 mg/kg to about 500 mg/kg, per day. The dose may be changed depending on the age, weight, susceptibility, or conditions of an individual.

In the method, the individual may be a mammal including a human.

### Advantageous Effects of Disclosure

A microorganism or a combination thereof or a culture or extract thereof according to an aspect may be used to reduce fat content or improve liver function.

A composition according to another aspect may be used to reduce fat content or improve liver function.

In a method according to another aspect, fat content may be efficiently reduced, or liver function may be efficiently improved.

### BRIEF DESCRIPTION OF DRAWINGS

FIGS. 1A and 1B are diagrams showing weight changes over time in a prophylactic model (FIG. 1A) and a treatment model (FIG. 1B) by administering two lactic acid bacteria in fatty liver-induced mice by high-fat diet.

FIGS. 2A and 2B are diagrams showing weight changes and lipid accumulation changes in a liver tissue in a prophylactic model (FIG. 2A) and a treatment model (FIG. 2B) by administering two lactic acid bacteria in fatty liver-induced mice by high-fat diet.

FIGS. 3A and 3B are diagrams showing a triglyceride content in a liver tissue in a prophylactic model (FIG. 3A) and a treatment model (FIG. 3B) by administering two lactic acid bacteria in fatty liver-induced mice by high-fat diet.

FIGS. 4A and 4B are diagrams showing AMPK activity in a liver tissue in a prophylactic model (FIG. 4A) and a treatment model (FIG. 4B) by administering two lactic acid bacteria in fatty liver-induced mice by high-fat diet.

FIGS. 5A and 5B are diagrams showing expression of fat oxidation-related genes and liposynthesis-related genes in a liver tissue in a prophylactic model (FIG. 5A) and a treatment model (FIG. 5B) by administering two lactic acid bacteria in fatty liver-induced mice by high-fat diet.

FIGS. 6A and 6B are diagrams showing weight changes in visceral adipose tissue in a prophylactic model (FIG. 6A) and a treatment model (FIG. 6B) by administering two lactic acid bacteria in fatty liver-induced mice by high-fat diet.

FIGS. 7A and 7B are diagrams showing AMPK activity in a visceral adipose tissue in a prophylactic model (FIG. 7A) and a treatment model (FIG. 7B) by administering two lactic acid bacteria in fatty liver-induced mice by high-fat diet.

FIGS. 8A and 8B are diagrams showing the amount of expression of fat oxidation-related genes and liposynthesis-related genes in a visceral adipose tissue in a prophylactic model (FIG. 8A) and a treatment model (FIG. 8B) by administering two lactic acid bacteria in fatty liver-induced mice by high-fat diet.

FIGS. 9A and 9B are diagrams showing a content of adiponectin in blood in a prophylactic model (FIG. 9A) and a treatment model (FIG. 9B) by administering two lactic acid bacteria in fatty liver-induced mice by high-fat diet.

### MODE OF DISCLOSURE

Hereinafter, the present disclosure will be described in more detail with reference to Examples. However, these Examples are for illustrative purposes only, and the present disclosure is not intended to be limited by these Examples.

### Example 1. Isolation of Strains

1. Isolation of Strains

Isolation of strains was carried out by taking 100 g of infant feces that were not exposed to traditional fermented foods and lactic acid bacteria prepared directly at home, diluting the infant feces in the sterile water, and homogenizing the infant feces with a stomacher for 5 minutes. The homogenized sample was serially diluted and plated on MRS (Difco, USA) agar plate medium containing bromophenol blue (Sigma, USA) and incubated at 37° C. for 2 to 3 days. The colonies that appeared were isolated by shape and color and pure-isolated again to obtain the final two strains. The pure-isolated lactic acid bacteria were subjected to a 16S rDNA phylogenetic analysis as shown in Example 1.2 to identify each lineage.

2. 16S rDNA Analysis

The selected lactic acid bacteria were respectively subjected to PCR with a primer set of 27F (SEQ ID NO: 3) and 1492R (SEQ ID NO: 4) and a genome of LMT15-14 and LMT19-1 as a template to obtain a 16S rDNA amplification product. The nucleotide sequence of the amplification product was confirmed through sequencing. As a result, the 16S rDNA of LMT15-14 and the 16S rDNA of LMT19-1 respectively have nucleotide sequences of SEQ ID NOS: 1 and 2. In addition, the nucleotide sequence of the 16S rDNA was interpreted using NCBI blast (http://www.ncbi.nlm.nih.gov/). Phylogenetic analysis showed that LMT15-14 was identical to Lactobacillus salivarius species, and LMT19-1 was identical to Lactobacillus plantarum species. The 16S rDNA of LMT15-14 and the 16S rDNA of LMT19-1 respectively had sequence identity with Lactobacillus salivarius species and Lactobacillus plantarum species of 99.9% and 99.9%, and thus, LMT15-14 and LMT19-1 strains were identified as new strains belonging to Lactobacillus salivarius species and Lactobacillus plantarum species, respectively. These two strains were each named Lactobacillus salivarius LMT15-14 and Lactobacillus plantarum LMT19-1, and these two strains were deposited with Deposit Nos. KCTC 14142BP and KCTC 14141BP on Feb. 21, 2020 at the Korea Collection for Type Cultures (KCTC) of the Korea Institute of Biotechnology.

### Example 2. Evaluation of Fatty Liver Inhibitory Efficacy by Lactic Acid Bacteria in Non-Alcoholic Fatty Liver Induced C57bl/6J Mouse Model by High-Fat Diet

1. Induction of Obesity in C57BL/6J Mice and Treatment of Lactic Acid Bacteria

To evaluate inhibitory efficacy on fatty liver caused by high-fat diet, fatty liver induced patterns were evaluated when lactic acid bacteria were administered in two models: a prophylactic model and a treatment model.

The animals used in the experiment were C57BL/6J mice, which was caused obesity with high-fat diet. The prophylactic model was 7 weeks old mice (males, 18 g to 22 g), and the treatment model was 4 weeks old mice (males, 13 g to 17 g) were purchased from Orient Bio. The mice were fed a regular diet (SAFE, France) for 1 week to adapt to the environment. Later, other groups except the normal control group of the prophylactic model were fed high-fat diet (Research diet, USA) and a positive control material and each lactic acid bacterium for 8 weeks once a day through a Sonde for oral administration directly to stomach to compare non-alcoholic fatty liver inducing pattern. The treatment model was induced to have non-alcoholic fatty liver by high-fat diet for 8 weeks. After 8 weeks, the treatment model was fed high-fat diet and a positive control material and each lactic acid bacterium for 16 weeks once a day through a Sonde for oral administration directly to stomach to compare non-alcoholic fatty liver inducing pattern. The groups (n=10) are a total of 8 groups, consisting of the following Table 1.

TABLE 1

| Group | Diet | Administered material | Concentration | |
|---|---|---|---|---|
| Normal control | Normal | PBS | N/A | |
| group | diet | | | |
| Negative control | High-fat | PBS | N/A | |
| group | diet | | | |
| Positive control | High-fat | Milk thistle | 100 | mg/kg/day |
| group | diet | | | |
| Positive control | High-fat | L. rhamnosus | 1 × 10⁹ | CFU/day |
| group | diet | GG KCTC 5033 | | |
| Experimental | High-fat | L. salivarius | 1 × 10⁹ | CFU/day |
| group | diet | LMT15-14 | | |
| Experimental | High-fat | L. plantarum | 1 × 10⁹ | CFU/day |
| group | diet | LMT19-1 | | |

Weight and dietary capacity were measured 1 time per week, and after the end of the experimental period, the experimental animals were fasted, and CO₂ gases were used to euthanize by inducing hypoxia and sleep. Plasma and tissue samples were stored at minus 80° C. until use.

2. Measurement of Weight Change in Non-Alcoholic Fatty Liver Induced C57bl/6J Mouse Model by High-Fat Diet

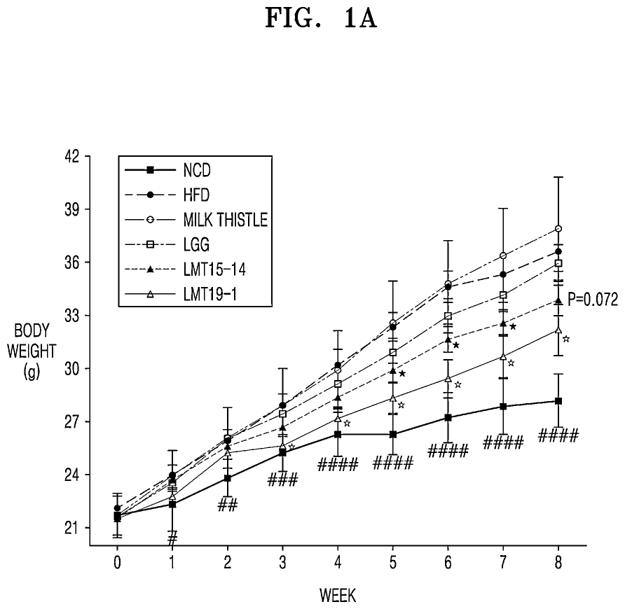

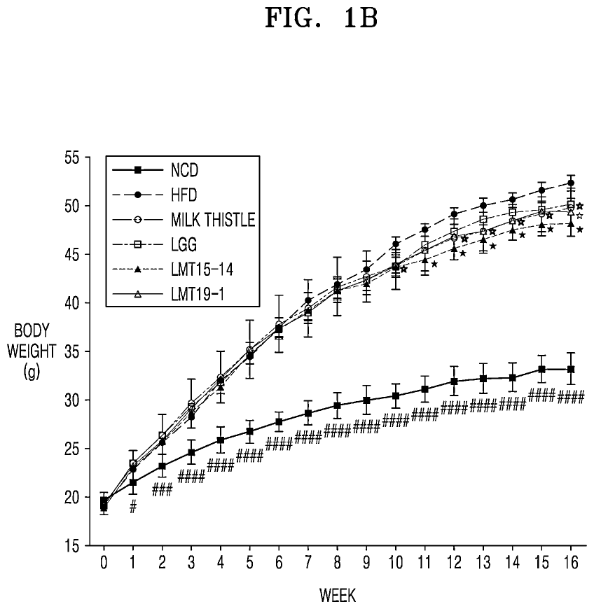

The weight of the experimental animals was measured at a certain time every day during the entire experimental period, and the results are shown in FIGS. 1A for the prophylactic model and 1B for the treatment model. FIG. 1 is a diagram showing weight changes over time in fatty liver-induced mice by high-fat diet by administering two selected lactic acid bacteria. In FIG. 1, the horizontal axis represents time (week), and the vertical axis represents the body weight.

As shown in FIG. 1, the group fed with fatty liver-inducing high-fat diet in the prophylactic model and treatment model gained weight. In case of the group administered orally with high-fat diet and lactic acid bacteria, as compared with the control group, in the prophylactic model, the weight reduced in L. salivarius LMT15-14 by 7.5%, and in L. plantarum LMT19-1 by 12.1%. In the treatment model, the weight reduced in L. salivarius LMT15-14 by 7.9%, and in L. plantarum LMT19-1 by 5.6%.

3. Analysis of Liver Tissue Change in Non-Alcoholic Fatty Liver Induced C57bl/6J Mouse Model by High-Fat Diet

(1) Weighing of Liver Tissue

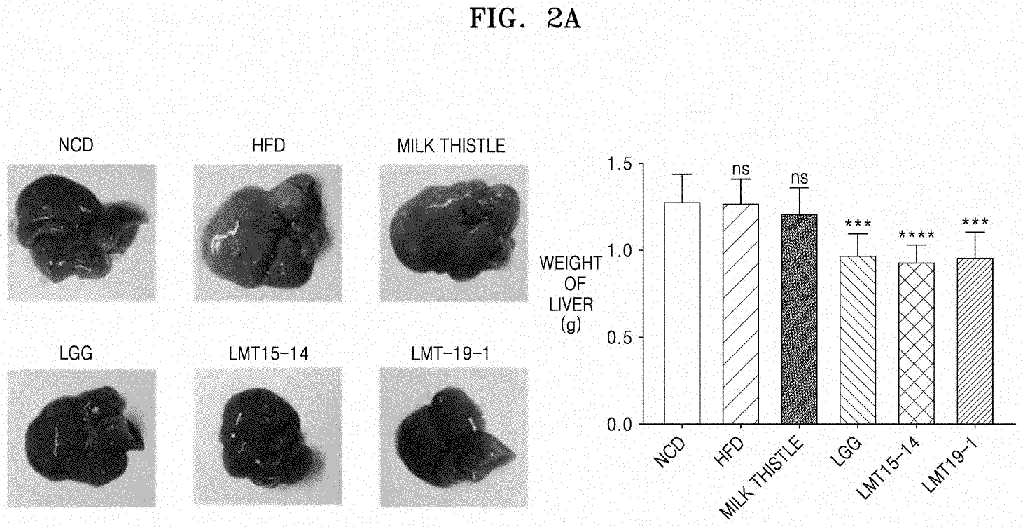

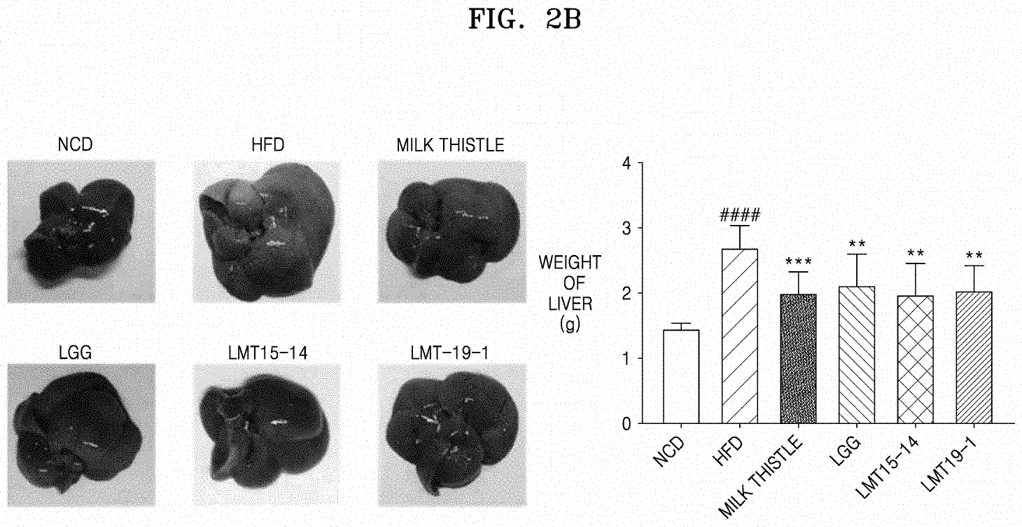

The effect of improving fatty liver by administering lactic acid bacteria in a non-alcoholic fatty liver induced model by high-fat diet was evaluated. After the end of the experimental period of the prophylactic model and the treatment model, liver tissue was extracted from mice of each group and weighed. The results are shown in FIG. 2A for the prophylactic model and FIG. 2B for the treatment model.

As shown in FIG. 2, upon identifying the weight of liver tissue, in case of the group administered orally with high-fat diet and lactic acid bacteria, as compared with the control group, in the prophylactic model, the weight reduced in L. salivarius LMT15-14 by 26.6%, and in L. plantarum LMT19-1 by 24.6%. In the treatment model, the weight reduced in L. salivarius LMT15-14 by 27.8%, and in L. plantarum LMT19-1 by 24.9%.

(2) Identification of Triglyceride Content in Liver Tissue

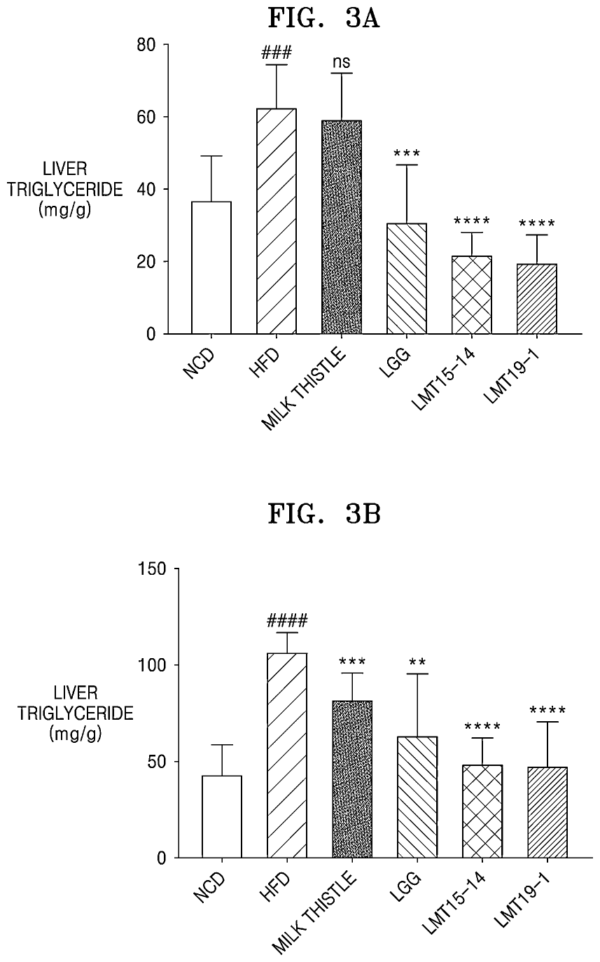

The content of triglyceride in liver was measured to identify the effect of improving fatty liver by administering lactic acid bacteria in a non-alcoholic fatty liver induced model by high-fat diet. Liver tissue samples from mice of each group were heated at 100° C. for 5 minutes using 5% NP-40 (BioVision, USA) and cooled at room temperature, and then this cycle was repeated 3 times. After the repetition, only the supernatant was obtained, and the content of triglycerides was measured by measuring an absorbance at 570 nm using a spectrophotometer using a triglyceride quantitative kit (BioVision, USA). The results thereof are shown in FIGS. 3A for the prophylactic model and 3B for the treatment model.

As shown in FIG. 3, upon identifying the content of triglycerides in liver, in case of the group administered orally with high-fat diet and lactic acid bacteria, as compared with the control group, in the prophylactic model, the weight reduced in L. salivarius LMT15-14 by 65.4%, and in L. plantarum LMT19-1 by 68.7%. In the treatment model, the weight reduced in L. salivarius LMT15-14 by 54.4%, and in L. plantarum LMT19-1 by 55.5%. The group administered lactic acid bacteria was found that the triglyceride content in liver tissue was significantly lower than the control group, which was expected that lactic acid bacteria improved non-alcoholic fatty liver by promoting degradation of triglycerides and inhibiting synthesis of triglycerides in the liver.

(3) Confirmation of AMP-Activated Protein Kinase (AMPK) Activation in Liver Tissue

AMPK is a protein that detects the state of energy in cells and regulates degradation and synthesis of sugars, fats, and cholesterol in liver, muscle, and adipose tissue associated with energy metabolism. That is, activation of AMPK, as a substance, which promotes absorption of sugars and oxidation of fats in cells, increases the oxidation of lipids and reduces triglyceride levels in liver tissue.

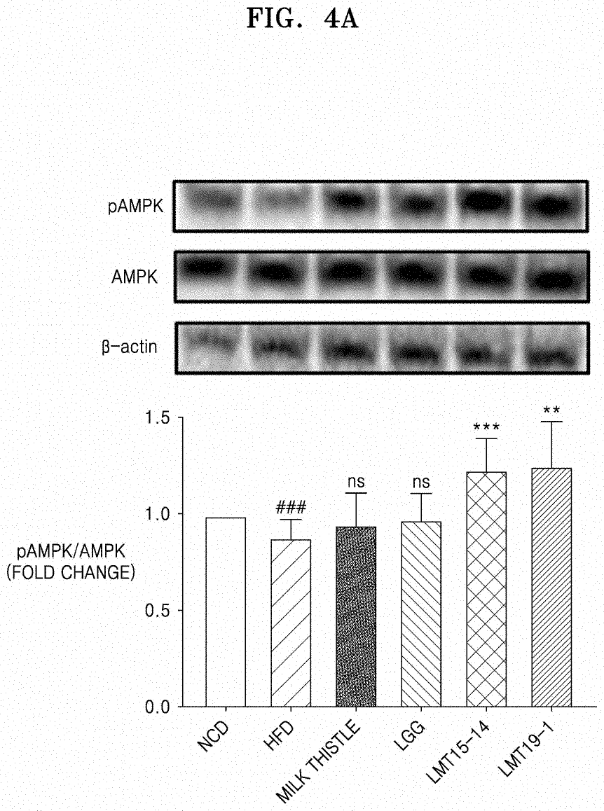

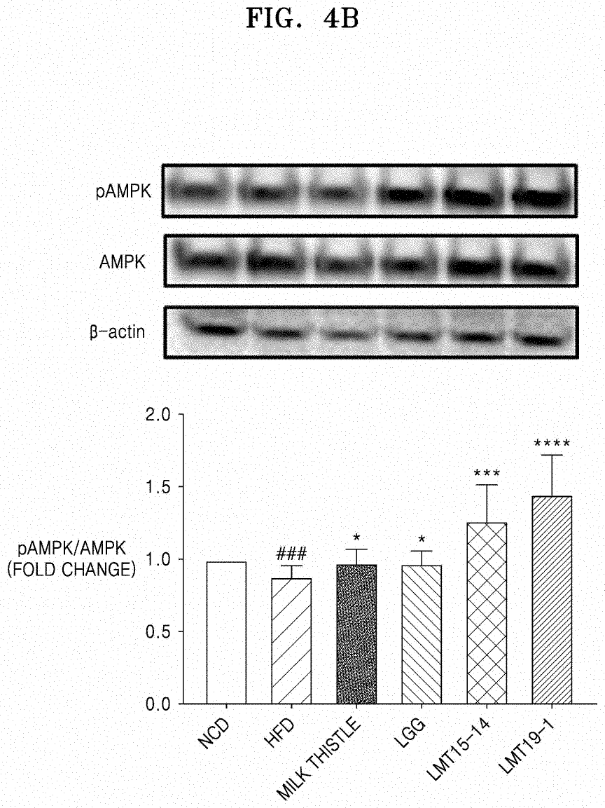

The level of activation of AMPK in liver tissue was identified to confirm the effect of improving fatty liver by administering lactic acid bacteria in a non-alcoholic fatty liver induced model by high-fat diet. That is, liver tissue samples obtained from mice of each group were obtained using PRO-PRE-P (Intron, Korea), a protein extraction solution. The extracted protein was quantified by Bradford assay (Bio-Rad, USA), then separated through electrophoresis in an SDS-polyacrylamide gel (Invitrogen, USA), and then transferred to a PVDF membrane (polyvinylidene difluoride membrane, Bio-Rad, USA). The protein-transferred PVDF membrane was blocked with a TBST 0.1% solution containing 5% BSA for 1 hour at room temperature, and then reacted for 18 hours at 4° C. with primary antibodies anti-p-AMPK, anti-AMPK, and anti-β-actin antibodies (1:1,000, Cell Signaling, USA). Once the reaction was complete, the resultant was washed with a TBST 0.1% solution and reacted at room temperature for 1 hour with a secondary antibody anti-rabbit IgG HRP-linked antibody (1:2,000, Cell Signaling, USA), and then washed with TBST 0.1%. After washing, the resultant was reacted with an ECL solution (Thermo Fisher Scientific, USA), and measurement was performed by using Chemi-doc (Bio-Rad, USA). The results thereof for the prophylactic model are shown in FIG. 4A and the results thereof for the treatment model are shown in FIG. 4B.

As shown in FIG. 4, upon identifying activation of AMPK in liver tissue, in case of the group administered orally with high-fat diet and lactic acid bacteria, as compared with the control group, in the prophylactic model, the phosphorylation of AMPK increased in L. salivarius LMT15-14 by 39.7%, and in L. plantarum LMT19-1 by 42.1%. In the treatment model, phosphorylation of AMPK increased in L. salivarius LMT15-14 by 45.4%, and in L. plantarum LMT19-1 by 66.0%. This result suggests that as phosphorylated AMPK increases, activation of fat oxidation in the liver increases, which may inhibit non-alcoholic fatty liver by regulating liposynthesis.

(4) Identification of Significant Differences in Fat Oxidation and Biomarker Gene Expression Related to Liposynthesis of Liver Tissue

In order to confirm the effect of improving fatty liver by administering lactic acid bacteria in a non-alcoholic fatty liver induced model by high-fat diet, liver tissue samples obtained from mice of each group were used to measure the difference in the expression of the fat oxidation-related genes PPAR-α and CPT1 and the liposynthesis-related genes SREBP-1c and FAS in the liver tissue through real-time PCR.

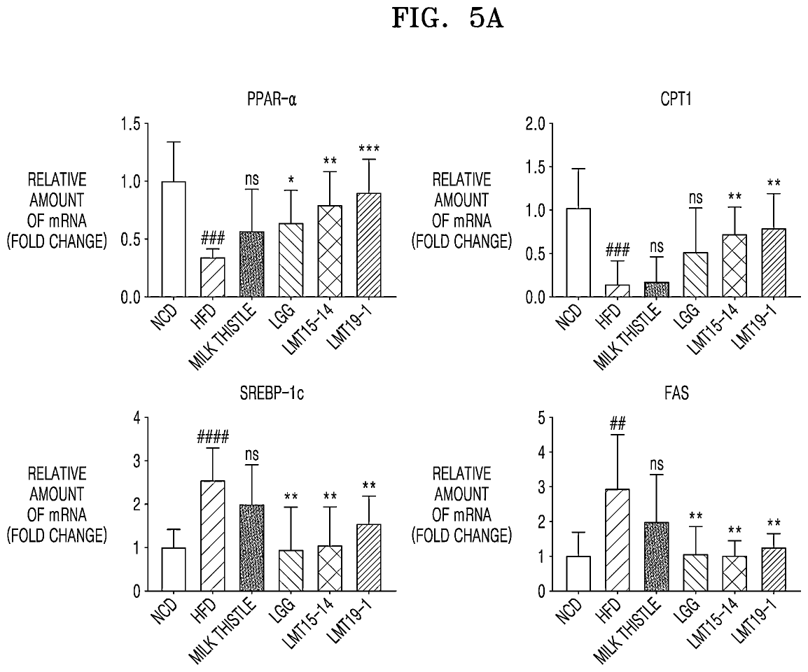

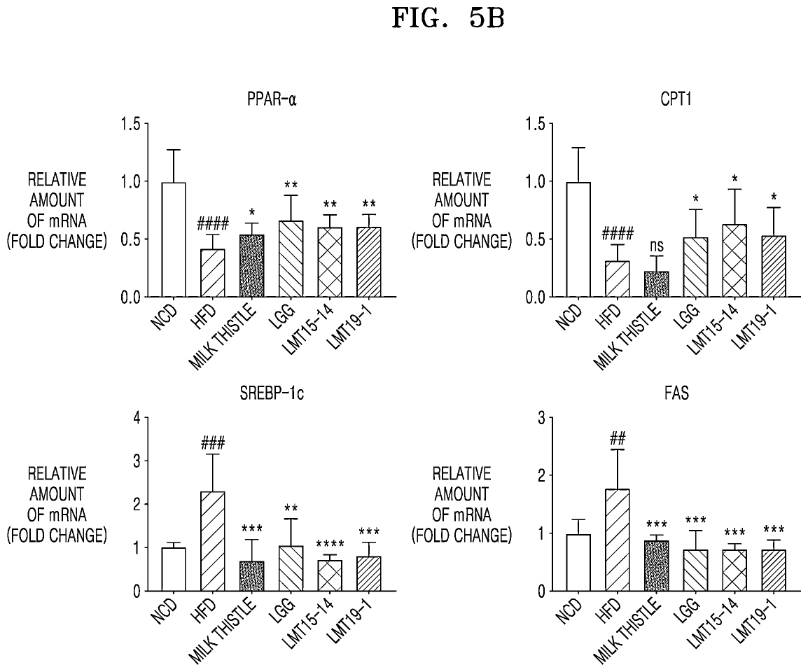

That is, RNA was extracted from liver tissue samples obtained from mice of each group by using the RNA extraction kit, AccuPrep® Universal RNA Extraction Kit (Bioneer, Korea). Later, after obtaining DNA complementary to RNA using RocketScript Cycle RT Premix (Bioneer, South Korea), expression of fat oxidation-related genes (PPAR-α and CPT1) and liposynthesis-related genes (SREBP-1c and FAS) was confirmed using SYBR green (Takara, Japan) and the primers shown in Table 2 below. The results thereof for the prophylactic model are shown in FIG. 5A and the results thereof for the treatment model are shown in FIG. 5B.

TABLE 2

| No. | Type | Gene | Primer | Sequence ID No. |
|---|---|---|---|---|
| 1 | Mouse | PPAR-α | Forward | 5 |
| | | | Reverse | 6 |
| 2 | | CPT1 | Forward | 7 |
| | | | Reverse | 8 |
| 3 | | SREBP-1c | Forward | 9 |
| | | | Reverse | 10 |
| 4 | | FAS | Forward | 11 |
| | | | Reverse | 12 |
| 5 | | GAPDH | Forward | 13 |
| | | | Reverse | 14 |

As shown in FIG. 5, upon identifying the amount of expression of fat oxidation-related genes, PPAR-α and CPT1, in liver tissue, in case of the group administered orally with high-fat diet and lactic acid bacteria, as compared with the control group, in the prophylactic model, the amount of expression increased in L. salivarius LMT15-14 by 131.3%, 438.1%, and in L. plantarum LMT19-1 by 163.6%, 494.9%. In the treatment model, the amount of expression increased in L. salivarius LMT15-14 by 43.5%, 102.2%, and in L. plantarum LMT19-1 by 44.2%, 69.2%. In addition, upon identifying the amount of expression of liposynthesis-related genes, SREBP-1c and FAS, in liver tissue, as compared with the control group, in the prophylactic model, the amount of expression decreased in L. salivarius LMT15-14 by 58.0%, 65.8%, and in L. plantarum LMT19-1 by 39.2%, 57.7%. In the treatment model, the amount of expression decreased in L. salivarius LMT15-14 by 69.1%, 60.1%, and in L. plantarum LMT19-1 by 65.7%, 60.1%. Therefore, fatty liver may be inhibited through increased fat oxidation and inhibition of liposynthesis within administration time of the lactic acid bacteria.

4. Analysis of Visceral Adipose Tissue Change in Non-Alcoholic Fatty Liver Induced C57bl/6J Mouse Model by High-Fat Diet

(1) Weighing of visceral adipose tissue

Normally, 80% of liver tissue fatty acids are introduced into liver tissue through the circulatory system after triglycerides in adipose tissue are degraded into fatty acids, 15% are absorbed through the digestive system after a meal and then introduced into liver tissue through the circulatory system, and the remaining 5% is newly produced through the fatty acid neoplastic process (de novo lipogenesis) of liver tissue. Thus, an increased influx of fatty acids from adipose tissue is closely related to the formation of excess fatty liver in liver tissue.

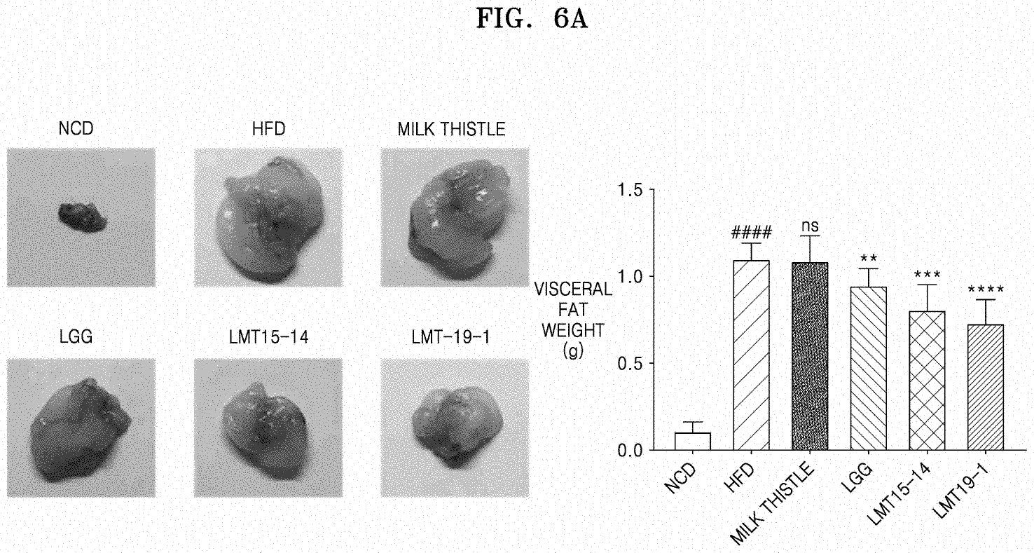

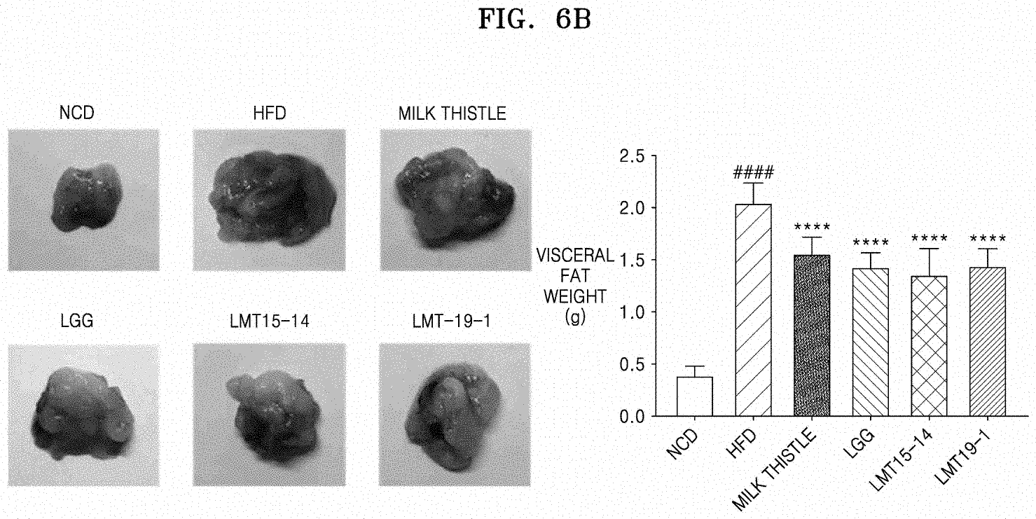

The effect of inhibiting visceral adipose tissue by administering lactic acid bacteria in a non-alcoholic fatty liver induced model by high-fat diet was evaluated. After the end of the experimental period of the prophylactic model and the treatment model, visceral adipose tissue from mice of each group, that is, the fat present in the abdominal side abdominal cavity, which is organically present between intestines except the subcutaneous fat, was extracted and weighed. The results are shown in FIG. 6A for the prophylactic model and FIG. 6B for the treatment model.

As shown in FIG. 6, upon identifying the weight of visceral adipose tissue, in case of the group administered orally with high-fat diet and lactic acid bacteria, as compared with the control group, in the prophylactic model, the weight reduced in L. salivarius LMT15-14 by 27.1%, and in L. plantarum LMT19-1 by 33.9%. In the treatment model, the weight reduced in L. salivarius LMT15-14 by 33.7%, and in L. plantarum LMT19-1 by 24.6%.

(2) Identification of Activation of AMPK in Visceral Adipose Tissue

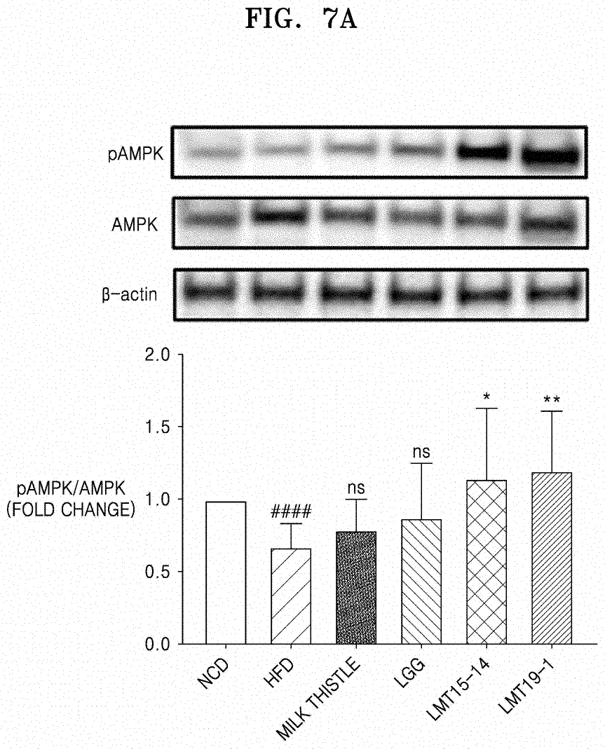

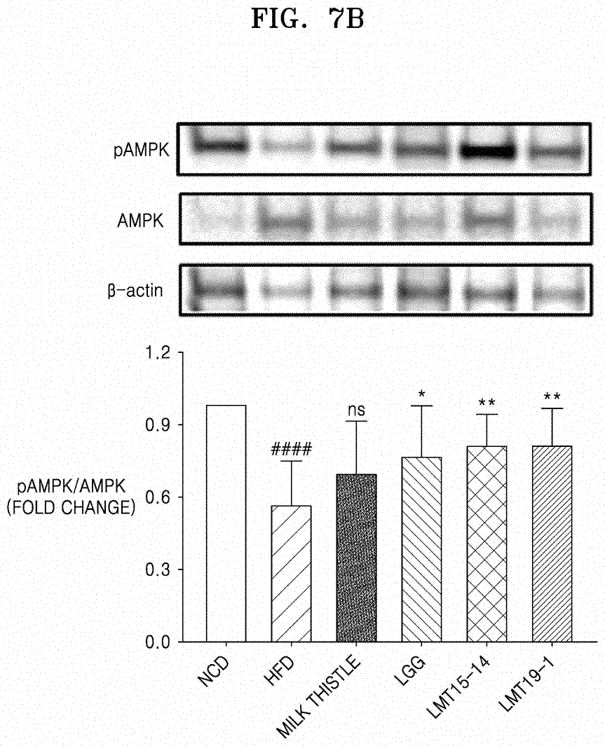

The level of activation of AMPK in visceral adipose tissue was identified to confirm the effect of improving fatty liver by administering lactic acid bacteria in a non-alcoholic fatty liver induced model by high-fat diet. Visceral adipose tissue samples obtained from mice of each group were used in the same manner as in Example 3.3. The results are shown in FIG. 7A for the prophylactic model and FIG. 7B for the treatment model.

As shown in FIG. 7, upon identifying activation of AMPK in visceral adipose tissue, in case of the group administered orally with high-fat diet and lactic acid bacteria, as compared with the control group, in the prophylactic model, the phosphorylation of AMPK increased in L. salivarius LMT15-14 by 73.0%, and in L. plantarum LMT19-1 by 80.8%. In the treatment model, phosphorylation of AMPK increased in L. salivarius LMT15-14 by 44.8%, and in L. plantarum LMT19-1 by 44.9%. This result suggests that as phosphorylated AMPK increases, activation of fat oxidation in the adipose increases, which may regulate liposynthesis and reduce introduction of fatty acid into liver.

(3) Identification of Significant Differences in Fat Oxidation and Biomarker Gene Expression Related to Liposynthesis of Visceral Adipose Tissue

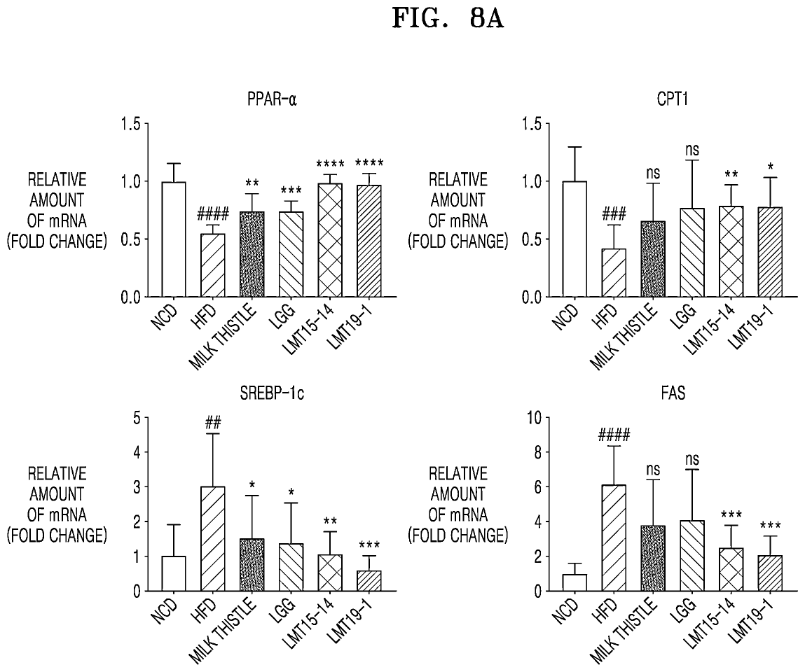

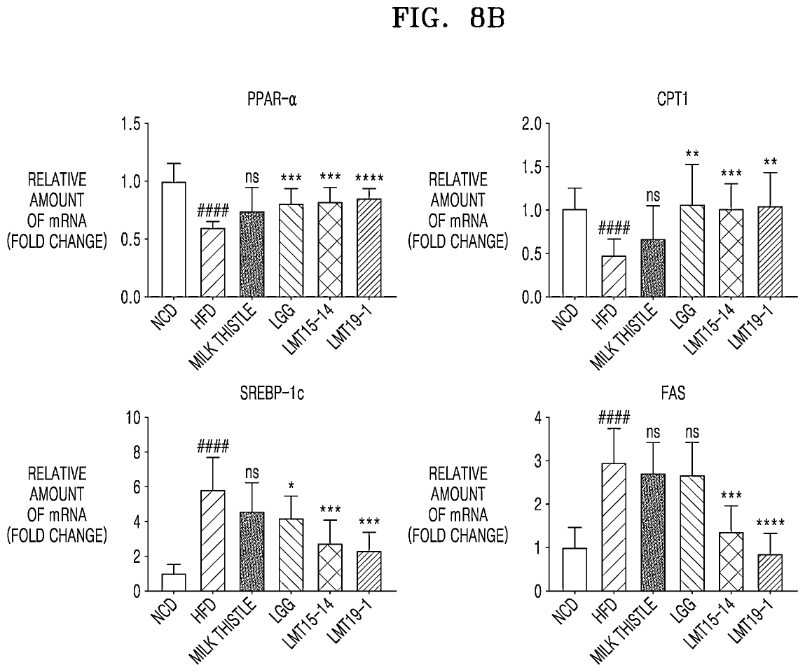

In order to confirm the effect of improving fatty liver by administering lactic acid bacteria in a non-alcoholic fatty liver induced model by high-fat diet, the difference in the expression of the fat oxidation-related genes PPAR-α and CPT1 and the liposynthesis-related genes SREBP-1c and FAS in the liver tissue were measured through real-time PCR. Visceral adipose tissue samples obtained from mice of each group were used in the same manner as in Example 3.4. The results are shown in FIG. 8A for the prophylactic model and FIG. 8B for the treatment model.

As shown in FIGS. 8A and 8B, upon identifying the amount of expression of fat oxidation-related genes, PPAR-α and CPT1, in visceral adipose tissue, in case of the group administered orally with high-fat diet and lactic acid bacteria, as compared with the control group, in the prophylactic model, the amount of expression increased in L. salivarius LMT15-14 by 78.8%, 86.8%, and in L. plantarum LMT19-1 by 76.6%, 83.0%. In the treatment model, the amount of expression increased in L. salivarius LMT15-14 by 36.9%, 112.3%, and in L. plantarum LMT19-1 by 41.7%, 117.5%. In addition, upon identifying the amount of expression of liposynthesis-related genes, SREBP-1c and FAS, in visceral adipose tissue, as compared with the control group, in the prophylactic model, the amount of expression decreased in L. salivarius LMT15-14 by 65.5%, 59.1%, and in L. plantarum LMT19-1 by 80.7%, 67.1%. In the treatment model, the amount of expression decreased in L. salivarius LMT15-14 by 53.4%, 53.7%, and in L. plantarum LMT19-1 by 59.3%, 71.1%. Therefore, upon administration, the lactic acid bacteria may inhibit the synthesis of triglycerides through increased fat oxidation and inhibition of liposynthesis of adipose tissue to reduce introduction of fatty acids into the liver to thereby inhibit fatty liver.

5. Identification of Adiponectin in Non-Alcoholic Fatty Liver Induced C57bl/6J Mouse Model by High-Fat Diet

Adiponectin is a hormone secreted by adipose tissue that affects AMPK activity and PPARα activity, affecting fat regulation. In obese patients, the amount of adiponectin in the blood decreases, and the decrease in body fat inhibits fatty liver by increasing adiponectin production, thereby promoting the β-oxidation of fatty acids. Adiponectin may be used as an indicator of fat accumulation because the expression amount and blood concentration are reduced when body fat is accumulated excessively.

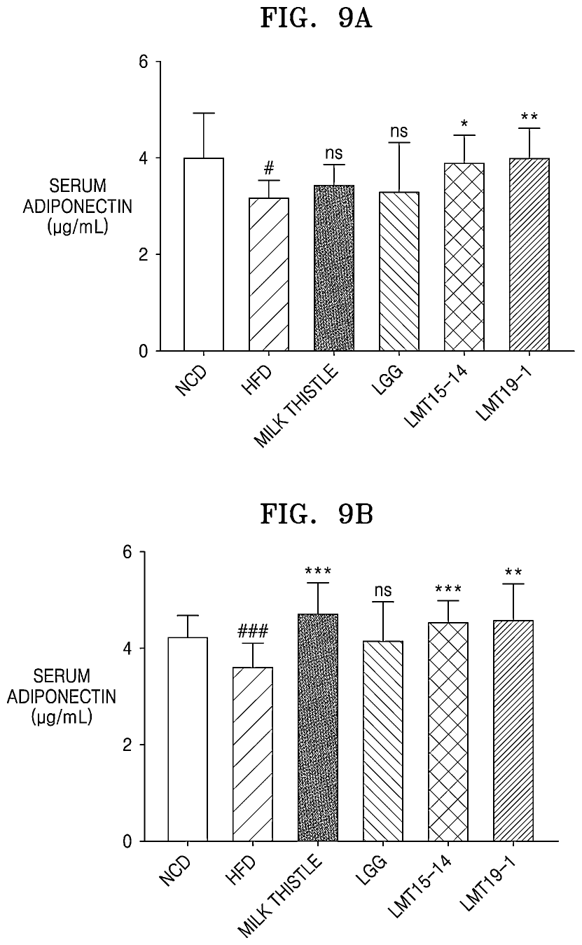

To measure the content of adiponectin, a hormone that affects fatty acid activation, blood samples taken from mice of each group were collected in a tube, and serum was separated by centrifugation. The separated serum was used in measuring the adiponectin content by using Adiponectin (mouse) ELISA kit (Adipogen Inc, Korea), and the results were shown in FIG. 9A for the prophylactic model and FIG. 9B for the treatment model.

As shown in FIGS. 9A and 9B, upon identifying the content of adiponectin in blood, in case of the group administered orally with high-fat diet and lactic acid bacteria, as compared with the control group, in the prophylactic model, adiponectin increased in L. salivarius LMT15-14 by 22.4%, and in L. plantarum LMT19-1 by 26.7%. In the treatment model, adiponectin increased in L. salivarius LMT15-14 by 25.6%, and in L. plantarum LMT19-1 by 26.3%. Therefore, as adiponectin increases, it is possible to inhibit fatty liver generation by increasing the activation of AMPK, which is involved in the β-oxidation of fatty acids.

### Example 3. Investigation of the Morphological and Fermentation Properties of Strains

1. Bacteriological Character Analysis

2 types of lactic acid bacteria, which are effective in inhibiting non-alcoholic fatty liver, L. salivarius LMT15-14 and L. plantarum LMT19-1 were cultured in MRS plate medium. Then, the form of colony was observed, and the forms of colonies are shown in Table 3.

TABLE 3

| | LMT15-14 | LMT19-1 |
|---|---|---|
| Form | Circular | Circular |
| Size | 1.5 mm | 1 mm |
| Color | Cream color | Cream color |
| Opacity | Opaque | Opaque |
| Bumps | Protruding | Protruding |
| Surface | Smooth | Smooth |
| Aerobic growth | + | + |
| Anaerobic growth | + | + |

2. Sugar Fermentation Properties of Selected Lactic Acid Strains

The sugar fermentation properties were investigated according to the suppliers experimental method using API 50 CHL kits (Biomerieux, France). 2 types of lactic acid bacteria, which are effective in inhibiting non-alcoholic fatty liver, L. salivarius LMT15-14 and L. plantarum LMT19-1, were subjected to investigation of sugar fermentation properties. The results thereof are shown in Table 4.

TABLE 4

| | LMT15-14 | LMT19-1 |
|---|---|---|
| Glycerol | − | − |
| Erythritol | − | − |
| D-Arabinose | − | − |
| L-Arabinose | − | + |
| D-Ribose | − | + |
| D-Xylose | − | − |
| L-Xylose | − | − |
| D-Adonitol | − | − |
| Methyl-β D-Xylopyranoside | − | − |
| D-Galactose | + | + |
| D-Glucose | + | + |
| D-Fructose | + | + |
| D-Mannose | + | + |
| L-Sorbose | − | − |
| L-Rhamnose | − | − |
| Dulcitol | − | − |
| Inositol | − | − |
| Mannitol | + | + |
| D-Sorbitol | + | + |
| Methyl α D-mannopyranoside | − | + |
| Methyl α D-glucopyranoside | − | − |
| N-Acetylglucosamine | + | + |
| Amygdalin | − | + |
| Arbutin | − | + |
| Esculin | + | + |
| Salicin | − | + |
| D-Cellobiose | − | + |
| D-Maltose | + | + |
| D-Lactose | + | + |
| D-Melibiose | + | + |
| D-Saccharose | + | + |
| D-Trehalose | − | + |
| Inulin | − | − |
| D-Melezitose | − | + |
| D-Raffinose | + | + |
| Amidon | − | − |
| Glycogen | − | − |
| Xylitol | − | − |
| Gentiobiose | − | + |
| D-Turanose | − | + |
| D-Lyxose | − | − |
| D-Tagatose | − | − |
| D-Fucose | − | − |
| L-Fucose | − | − |
| D-Arabitol | − | − |
| L-Arabitol | − | − |
| Potassium Gluconate | − | + |
| Potassium 2-Ketogluconate | − | − |
| Potassium 5-Ketogluconate | − | − |

### Example 7. Stability

1. Investigation of Acid Resistance of Lactic Acid Bacteria

In order for lactic acid bacteria to be effective as probiotics in intestines, lactic acid bacteria must pass through stomach at a low pH after ingestion. To investigate the acid resistance of lactic acid bacteria, after inoculation, the sterile MRS liquid medium was incubated at 37° C. for 18 hours and then adjusted to pH 2.5 with HCl to inoculate the lactic acid bacteria in sterile MRS liquid medium and incubated at 37° C. for 2 hours. Samples immediately after lactic acid bacteria inoculation and after 2 hours of incubation were recovered, diluted in MRS liquid medium, smeared on MRS plate medium, incubated at 37° C. for 24 hours, and then the number of colonies on the plate medium was counted to measure the number of lactic acid bacteria. The results thereof are shown in Table 5.

TABLE 5

Lactic acid bacteria (CFU/mL)

| | LMT15-14 | LMT19-1 |
|---|---|---|
| MRS (pH 6.8) | 3.2 × 10⁹ | 4.6 × 10⁹ |
| MRS (pH 2.5) | 2.7 × 10⁹ | 4.5 × 10⁹ |

### Example 7. Stability

1. Investigation of Acid Resistance of Lactic Acid Bacteria

In order for lactic acid bacteria to be effective as probiotics in intestines, lactic acid bacteria must pass through stomach at a low pH after ingestion. To investigate the acid resistance of lactic acid bacteria, after inoculation, the sterile MRS liquid medium was incubated at 37° C. for 18 hours and then adjusted to pH 2.5 with HCl to inoculate the lactic acid bacteria in sterile MRS liquid medium and incubated at 37° C. for 2 hours. Samples immediately after lactic acid bacteria inoculation and after 2 hours of incubation were recovered, diluted in MRS liquid medium, smeared on MRS plate medium, incubated at 37° C. for 24 hours, and then the number of colonies on the plate medium was counted to measure the number of lactic acid bacteria. The results thereof are shown in Table 6.

TABLE 6

Lactic acid bacteria (CFU/mL)

| | LMT15-14 | LMT19-1 |
|---|---|---|
| MRS (pH 6.8) | 3.2 × 10⁹ | 4.6 × 10⁹ |
| MRS (pH 2.5) | 2.7 × 10⁹ | 4.5 × 10⁹ |

As a result, 2 types of lactic acid bacteria, which are effective in inhibiting non-alcoholic fatty liver, L. salivarius LMT15-14 and L. plantarum LMT19-1, respectively had a viability of 84.4% and 97.8%, confirming a high viability for acid at pH 2.5. The characteristic of these lactic acid bacteria is that as the optimal number of 50% or greater of lactic acid bacteria was maintained at a pH lower than pH 3 close to the physiological pH of the stomach, the number of lactic acid bacteria may be maintained stably even at low pH due to gastric acid secretion, and the intestinal reach rate when ingested may be expected to be very high.

2. Investigation of Bile Resistance of Lactic Acid Bacteria

In order to investigate the bile resistance of lactic acid bacteria, experiments were carried out in the following method. Lactic acid bacteria were incubated at 37° C. for 18 hours after inoculation in sterile MRS liquid medium, and the bile acid concentration in the intestinal tract was around 0.1 (w/v) %. Thus, the lactic acid bacteria were inoculated in MRS liquid medium containing 0.3 (w/v) % bile salts (Sigma, USA) and incubated at 37° C. for 2 hours. Samples immediately after lactic acid bacteria inoculation and after 2 hours of incubation were recovered, diluted in MRS liquid medium, smeared on MRS plate medium, incubated at 37° C. for 24 hours, and then the number of colonies on the plate medium was counted to measure the number of lactic acid bacteria. The results thereof are shown in Table 7.

TABLE 7

Lactic acid bacteria (CFU/ml)

| | LMT15-14 | LMT19-1 |
|---|---|---|
| MRS | 3.2 × 10⁹ | 4.6 × 10⁹ |
| MRS (0.3% bile salt) | 2.7 × 10⁷ | 3.4 × 10⁹ |

As a result, 2 types of lactic acid bacteria, which are effective in inhibiting non-alcoholic fatty liver, L. salivarius LMT15-14 and L. plantarum LMT19-1 respectively had the number of lactic acid bacteria of 0.8% and 73.9%, respectively. In particular, L. plantarum LMT19-1 maintained a proper number of lactic acid bacteria of at least 50% at 0.3%, which is higher than 0.1%, which is similar to the actual concentration in the intestine. Therefore, it may be a basis for predicting that the lactic acid bacteria may survive sufficiently in intestines of a human body or animal, and that an intestinal reach rate may be very high.

1. Lactobacillus salivarius LMT15-14 (Accession No. KCTC14142BP) having activity of inhibiting triglycerides, promoting fat oxidation, and inhibiting liposynthesis.

2. Lactobacillus plantarum LMT19-1 (Accession No. KCTC14141BP) having activity of inhibiting triglycerides, promoting fat oxidation, and inhibiting liposynthesis.

3. A pharmaceutical composition, for improving liver function or preventing or treating obesity-related disease, containing a microorganism of claim 1 or a culture or extract thereof, or mixtures thereof, as an active ingredient.

4. The pharmaceutical composition of claim 3, wherein the obesity-related disease is at least one selected from the group consisting of non-alcoholic fatty liver, type 2 diabetes, hyperlipidemia, cardiovascular disease, atherosclerosis, lipid-related metabolic syndrome, and obesity.

5. A food composition, for improving liver function or preventing or improving obesity-related disease, containing a microorganism of claim 1 or a culture or extract thereof, or mixtures thereof, as an active ingredient.

6. The food composition of claim 5, wherein the obesity related disease is at least one selected from the group consisting of non-alcoholic fatty liver, type 2 diabetes, hyperlipidemia, cardiovascular disease, atherosclerosis, lipid-related metabolic syndrome, and obesity.

7. A pharmaceutical composition for improving liver function or preventing or treating obesity-related disease, containing a microorganism of claim 2 or a culture or extract thereof, or mixtures thereof, as an active ingredient.

8. A food composition for improving liver function or preventing or improving obesity-related disease, containing a microorganism of claim 2 or a culture or extract thereof, or mixtures thereof, as an active ingredient.


SEQUENCE LISTING (14 sequences)

SEQ ID NO: 1 - DNA, 1340 bp, Lactobacillus salivarius LMT15-14
ggcggacgggtgagtaacacgtgggtaacctgcctaaaagaaggggataacacttggaaacaggtgctaataccgtatatctctaaggatcgcatgatccttagatgaaagatggttctgctatcgcttttagatggacccgcggcgtattaactagttggtggggtaacggcctaccaaggtgatgatacgtagccgaactgagaggttgatcggccacattgggactgagacacggcccaaactcctacgggaggcagcagtagggaatcttccacaatggacgcaagtctgatggagcaacgccgcgtgagtgaagaaggtcttcggatcgtaaaactctgttgttagagaagaacacgagtgagagtaactgttcattcgatgacggtatctaaccagcaagtcacggctaactacgtgccagcagccgcggtaatacgtaggtggcaagcgttgtccggatttattgggcgtaaagggaacgcaggcggtcttttaagtctgatgtgaaagccttcggcttaaccggagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgaatgctaggtgttggagggtttccgcccttcagtgccgcagctaacgcaataagcattccgcctggggagtacgaccgcaaggttgaaactcaaaggaattgacgggggcccgcacaagcggtggagcatgtggtttaattcgaagcaacgcgaagaaccttaccaggtcttgacatcctttgaccacctaagagattaggctttcccttcggggacaaagtgacaggtggtgcatggctgtcgtcagctcgtgtcgtgagatgttgggttaagtcccgcaacgagcgcaacccttgttgtcagttgccagcattaagttgggcactctggcgagactgccggtgacaaaccggaggaaggtggggacgacgtcaagtcatcatgccccttatgacctgggctacacacgtgctacaatggacggtacaacgagtcgcaagaccgcgaggtttagctaatctcttaaagccgttctcagttcggattgtaggctgcaactcgcctacatgaagtcggaatcgctagtaatcgcgaatcagcatgtcgcggtgaatacgttcccgggccttgtacacaccgcccgtcacaccatgagagtttgtaacacccaaa

SEQ ID NO: 2 - DNA, 1308 bp, Lactobacillus plantarum LMT19-1
cacgtgggaaacctgcccagaagcgggggataacacctggaaacagatgctaataccgcataacaacttggaccgcaggtccgagtttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagag

SEQ ID NO: 3 - DNA, 20 bp, Artificial
  primer
agagtttgatcmtggctcag

SEQ ID NO: 4 - DNA, 19 bp, Artificial
  primer
ggttaccttgttacgactt

SEQ ID NO: 5 - DNA, 20 bp, Artificial
  primer
aagggcttctttcggcgaac

SEQ ID NO: 6 - DNA, 27 bp, Artificial
  primer
tgaccttgttcatgttgaagttcttca

SEQ ID NO: 7 - DNA, 24 bp, Artificial
  primer
acagtcggtgaggcctcttatgaa

SEQ ID NO: 8 - DNA, 24 bp, Artificial
  primer
tcttgctgcctgaatgtgagttgg

SEQ ID NO: 9 - DNA, 22 bp, Artificial
  primer
ggaggggtagggccaacggcct

SEQ ID NO: 10 - DNA, 22 bp, Artificial
  primer
catgtcttcgaaagtgcaatcc

SEQ ID NO: 11 - DNA, 20 bp, Artificial
  primer
cggtacgcgacggctgcctg

SEQ ID NO: 12 - DNA, 24 bp, Artificial
  primer
gctgctccacgaactcaaacaccg

SEQ ID NO: 13 - DNA, 21 bp, Artificial
  primer
catgtacgttgctatccaggc

SEQ ID NO: 14 - DNA, 21 bp, Artificial
  primer
ctccttaatgtcacgcacgat